(19) United States
(12) Patent Application Publication
(10) Pub. No.: US 2020/0282185 A1
(43) Pub. Date: Sep. 10, 2020

(54) GUIDEWIRE

(71) Applicant: ASAHI INTECC CO., LTD., Seto-shi (JP)

(72) Inventor: Kenta TSUGE, Seto-shi (JP)

(73) Assignee: ASAHI INTECC CO., LTD., Seto-shi (JP)

(21) Appl. No.: 16/881,094

(22) Filed: May 22, 2020

Related U.S. Application Data
(63) Continuation of application No. PCT/JP2018/003327, filed on Feb. 1, 2018.

(51) Int. Cl. A61M 25/09

(52) U.S. Cl. CPC A61M 25/09; A61M 2025/09083; A61M 2025/09175; A61M 2205/0266; A61M 2025/09133

(57) ABSTRACT

A guidewire includes, for example, a core shaft, a coil body having a distal end that is joined to the core shaft at a position spaced proximally from a distal end of the core shaft, and a coating agent covering an outer periphery of the core shaft and the coil body. The core shaft includes a bulge portion joined at a position spaced distally from the distal end of the coil body, and the coating agent covers the core shaft and the coil body from a distal side of the bulge portion to a proximal side of a proximal end of the coil body except for an interior of the coil body.

### CROSS REFERENCE TO RELATED APPLICATION

This is a Continuation of PCT/JP2018/003327 filed Feb. 1, 2018. The disclosure of the prior application is hereby incorporated by reference herein in its entirety.

### BACKGROUND

The disclosed embodiments relate to a medical device. In particular, the disclosed embodiments relate to a guidewire.

Conventionally, a variety of guidewires for guiding a catheter or the like that are used by being inserted into tubular organs such as a blood vessel, a gastrointestinal tract and a ureter or body tissues for treatment or examination have been developed. For example, Japanese Patent Application Publication No. 2004-41254 A discloses a guidewire comprising a contrast portion at a distal portion of a core member, and a synthetic resin coating layer on an outer periphery of the core member and the contrast portion (see FIG. 1 etc.). Further, a lubricious coating agent is applied to a surface of the synthetic resin coating layer described in Japanese Patent Application Publication No. 2004-41254 A.

As the guidewire described in Japanese Patent Application Publication No. 2004-41254 A includes a coil body being the contrast portion at the distal portion of the core member, and includes the synthetic resin coating layer on the outer periphery of the coil body, there is a problem that flexibility of a distal portion of the guidewire is impaired.

The synthetic resin coating layer and a lubricious coating agent formed on the surface of the synthetic resin coating layer (hereinafter, a synthetic resin coating layer and a lubricious coating agent are collectively referred to as “coating agent”) are likely to be peeled from the core member in the guidewire described in Japanese Patent Application Publication No. 2004-41254 A. Although the possibility of delamination has been slightly improved by the anchor effect due to an outer diameter of the contrast portion being larger than an outer diameter of the core member (hereinafter, referred to as “core shaft”), it was not sufficient yet.

The disclosed embodiments have been devised in response to the forgoing problems of the conventional technique and include a guidewire capable of ensuring flexibility of a distal portion of the guidewire and preventing the coating agent from peeling from the core shaft.

### SUMMARY

To address the foregoing problems, a guidewire according to the disclosed embodiments comprises a core shaft, a coil body having a distal end that is joined to the core shaft at a position spaced proximally from a distal end of the core shaft, and a coating agent covering an outer periphery of the core shaft and the coil body, wherein the core shaft includes a bulge portion joined at a position spaced distally from the distal end of the coil body, the coating agent covers the core shaft and the coil body from a distal side of a distal end of the bulge portion to a proximal side of a proximal end of the core shaft except for an interior of the coil body.

A guidewire of the disclosed embodiments can comprise a stranded wire joined to a distal portion of the core shaft, including a plurality of wires and extending distally from the distal end of the core shaft, wherein the coil body extends distal to the distal end of the core shaft and along the stranded wire, the coating agent covers an outer periphery of the stranded wire, the core shaft and the coil body, the bulge portion is joined to the stranded wire at a position spaced distally from a distal end of the coil body, and the coating agent covers the stranded wire, the core shaft and the coil body from the distal side of the distal end of the bulge portion to the proximal side of the proximal end of the core shaft except for the interior of the coil body.

The coating agent can penetrate between a plurality of wires included in the stranded wire.

According to the guidewire of the disclosed embodiments, as a guidewire comprises a core shaft, a coil body having a distal end that is joined to the core shaft at a position spaced proximally from a distal end of the core shaft, and a coating agent covering an outer periphery of the core shaft and the coil body, wherein the core shaft includes a bulge portion joined at a position spaced distally from the distal end of the coil body, and the coating agent covers the core shaft and the coil body from a distal side of a distal end of the bulge portion to a proximal side of a proximal end of the core shaft except for an interior of the coil body, it is possible to ensure flexibility of the coil body and a distal portion of the guidewire and prevent the coating agent from peeling from the core shaft and the coil body by the bulge portion and the coil body.

Further, when the guidewire comprises a stranded wire joined to a distal portion of the core shaft, including a plurality of wires and extending distally from the distal end of the core shaft, and wherein the coil body extends distal to the distal end of the core shaft and along the stranded wire, the coating agent covers an outer periphery of the stranded wire, the core shaft and the coil body, the bulge portion is joined to the stranded wire at a position spaced distally from a distal end of the coil body, and the coating agent covers the stranded wire, the core shaft and the coil body from the distal side of the distal end of the bulge portion to the proximal side of the proximal end of the coil body except for the interior of the coil body, it is possible to ensure more flexibility of the coil body, the stranded wire and a distal portion of the guidewire and further prevent the coating agent from peeling from the core shaft, the stranded wire and the coil body by the bulge portion and the coil body.

Furthermore, when the coating agent penetrates between a plurality of wires included in the stranded wire, it is possible to further prevent the coating agent from peeling from the stranded wire.

### BRIEF DESCRIPTION OF THE DRAWINGS

FIG. 1 is a schematic view of a guidewire of the disclosed embodiments.

FIG. 2 is a longitudinal sectional view of the guidewire of FIG. 1.

FIG. 3 is a schematic diagram of a guidewire of the disclosed embodiments.

FIG. 4 is a longitudinal sectional view of the guidewire of FIG. 3.

FIG. 5 is a longitudinal sectional view of a distal portion of a guidewire of the disclosed embodiments.

FIG. 6 is a longitudinal sectional view near a stranded wire included in the guidewire of FIG. 5.

### DETAILED DESCRIPTION OF EMBODIMENTS

Hereinafter, embodiments of the present invention will be described with reference to the drawings.

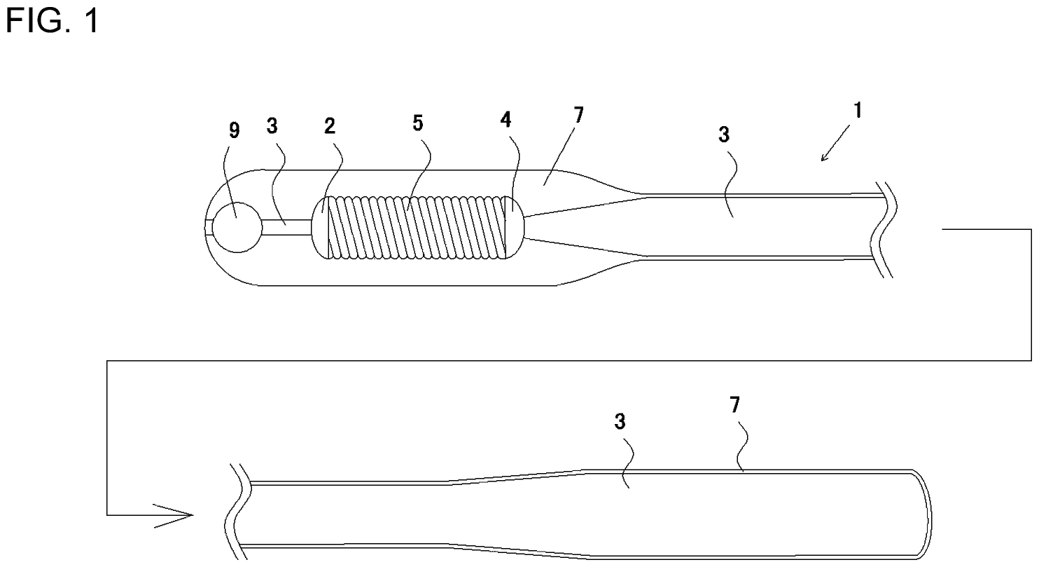

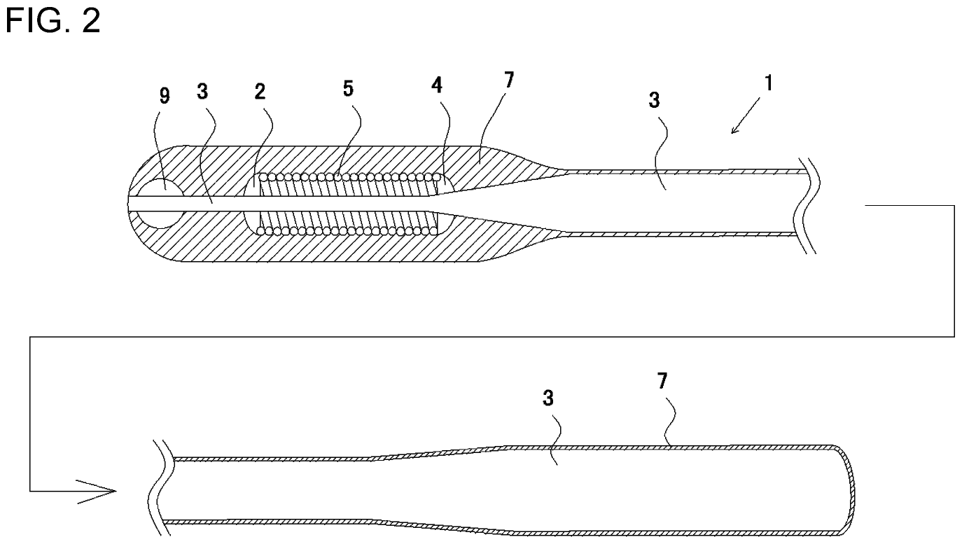

FIG. 1 is a schematic view of a guidewire of the disclosed embodiments, and FIG. 2 is a longitudinal sectional view of the guidewire of FIG. 1.

As shown in FIG. 1, a guidewire 1 of the disclosed embodiments includes a core shaft 3, a coil body 5 joined to the core shaft 3 at a position spaced proximally from a distal end of the core shaft 3, a bulge portion 9 joined to the core shaft 3 at a position spaced distally from a distal end of the coil body 5, and a coating agent (coating layer) 7 covering an outer periphery of the core shaft 3, the coil body 5 and the bulge portion 9.

The core shaft 3 is a rod-like member of circular cross-section, which is reduced in diameter toward a distal end from a proximal end of the core shaft 3, and is an elongate flexible member. Material of the core shaft 3 is not particularly limited as long as it is a biocompatible material such as stainless steel, Ni—Ti-based alloys, cobalt based alloys or the like.

The coil body 5 is joined to the core shaft at a position spaced proximally from a distal end of the core shaft 3. Specifically, a distal end of the coil body 5 is joined to the core shaft 3 by a brazing material 2, and a proximal end of the coil body 5 is joined to the core shaft 3 by a brazing material 4.

Incidentally, the brazing material 2 and the brazing material 4 are not particularly limited as long as they are each a biocompatible brazing material such as gold-tin brazing material, silver-tin brazing material or the like. The coil body 5 is a cylindrical hollow coil which is formed by winding a single metal wire or a plurality of metal wires. An outer diameter of the coil body 5 is larger than an outer diameter of the core shaft 3 in the position in which the coil body 5 is joined.

Material of the metal wire forming the coil body 5 is not particularly limited as long as it is a biocompatible material such as stainless steel, tungsten, Ni—Ti-based alloys or the like.

The bulge portion 9 is joined to the distal end of the core shaft 3 at a position spaced distally from the distal end of the coil body 5. An outer diameter of the bulge portion 9 is greater than an outer diameter of the core shaft 3 at a position at which the bulge portion 9 is joined to the core shaft 3. Material used for the bulge portion 9 is generally a brazing material similar to the brazing material 2 and the brazing material 4 joining the coil body 5 to the core shaft 3.

An area of the coating agent 7 is illustrated by hatching in FIG. 2. As shown in FIG. 2, the coating agent 7 covers the core shaft 3, the coil body 5 and the bulge portion 9 from a distal end of the guidewire 1 (which is at a distal side of the distal end of the bulge portion 9) to the proximal side of the proximal end of the coil body 5 except for the interior of the coil body 5.

It is to ensure the flexibility of the distal end of the guidewire 1 not to allow the coating agent 7 to penetrate inside the coil body 5.

The coating agent 7 can cover the core shaft 3 all the way to the proximal end of the core shaft 3 on a proximal side of the proximal end of the coil body 5 in order to reduce frictional resistance of the guidewire 1.

The coating agent 7 is preferably formed by low friction material, e.g. polyvinyl alcohol, polyvinyl pyrrolidone, polyethylene glycol, polyacrylamide, polyacrylic acid, sodium polyacrylate, poly (2-hydroxyethyl methacrylate), maleic anhydride copolymer, ethylene-vinyl alcohol copolymer, 2-methacryloyloxyethyl phosphorylcholine or a copolymer thereof, (2-hydroxyethyl methacrylate)-styrene block copolymer, various synthetic polypeptides, collagen, hyaluronic acid, cellulose-based polymer, and mixtures thereof.

Further, the coating agent 7 may be made of different materials depending on a position of the guidewire 1. For example, material of a distal side region from a middle position of the coil body 5 may be a coating agent 7 made of different materials from material of a proximal side region therefrom.

Because the guidewire 1 comprises a core shaft 3, a coil body 5 having a distal end that is joined to the core shaft 3 at a position spaced proximally from a distal end of the core shaft 3, and a coating agent 7 covering an outer periphery of the core shaft 3 and the coil body 5, wherein the core shaft 3 includes a bulge portion 9 joined at a position spaced distally from the distal end of the coil body 5, and the coating agent 7 covers the core shaft 3 and the coil body 5 from a distal side of a distal end of the bulge portion 9 to a proximal side of a proximal end of the coil body 5 except for an interior of the coil body 5, it is possible to ensure flexibility of the coil body 5 and a distal portion of the guidewire 1 and prevent the coating agent 7 from peeling from the core shaft 3 and the coil body 5 by the bulge portion 9 and the coil body 5.

The bulge portion 9 may be joined to the distal end of the core shaft 3 so that the distal end of the core shaft 3 does not protrude from the bulge portion 9. According to this configuration, it is possible to prevent the distal end of the core shaft 3 from protruding from the coating agent 7 and damaging to the body tissue.

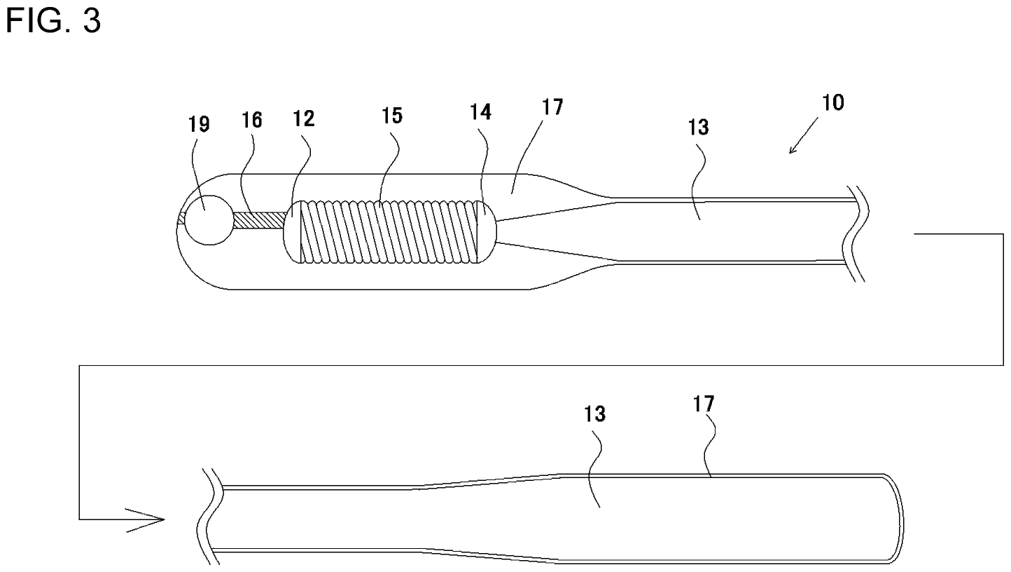

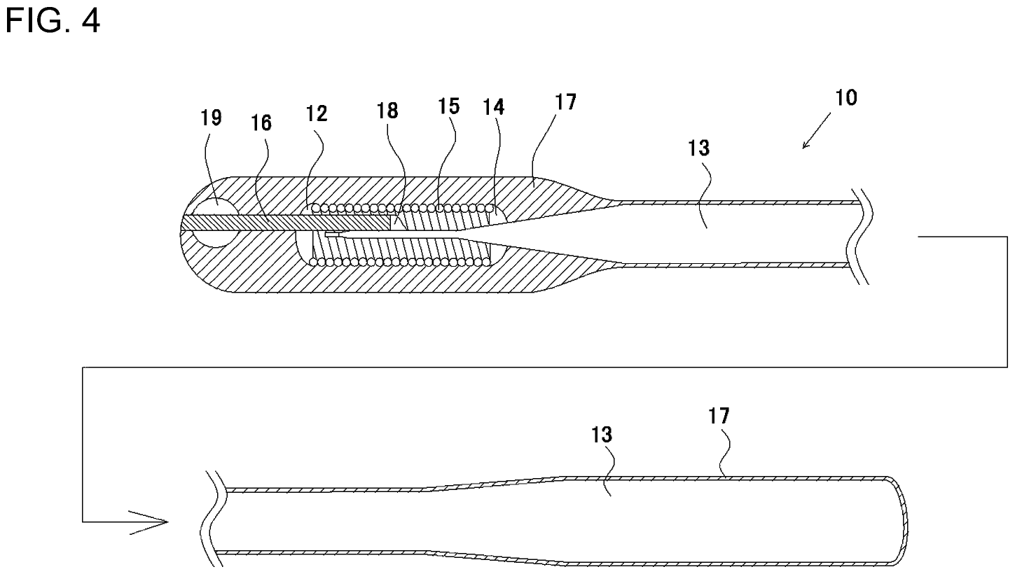

FIG. 3 is a schematic diagram of a guidewire of the disclosed embodiments, and FIG. 4 is a longitudinal sectional view of the guidewire of FIG. 3.

As shown in FIG. 3, a guidewire 10 of the disclosed embodiments includes a core shaft 13, a stranded wire 16 joined to a distal portion of the core shaft 13 so as to be located on a distal side of the core shaft 13 and including a plurality of wires, a coil body 15 extending distal to a distal end of the core shaft 13 and having a distal end joined to the stranded wire 16 at a position spaced proximally from a distal end of the stranded wire 16 and a proximal end joined to the core shaft 13, a coating agent 17 covering an outer periphery of the core shaft 13, the stranded wire 16 and the coil body 15, and a bulge portion 19 joined to the stranded wire 16 at a position spaced distally from a distal end of the coil body 5.

The core shaft 13 is a rod-like member of circular cross-section, which is reduced in diameter toward a distal end from a proximal end of the core shaft 13, and is an elongate flexible member similar to the core shaft 3. Material of the core shaft 13 is the same as that of the core shaft 3.

As shown in FIG. 4, the stranded wire 16 includes a proximal end joined to a distal portion of the core shaft 13 by a brazing material 18 inside the coil body 15, and extends distal to a distal end of the core shaft 13 and a distal end of the coil body 15. For example, the proximal end of the stranded wire 16 may be joined to the core shaft 13 at a position approximately halfway along the longitudinal length of the coil body 15.

Incidentally, the stranded wire 16 may be joined to both the coil body 15 and the core shaft 13 by the brazing material 18.

The coil body 15 includes a distal end joined to the stranded wire 16 and a proximal end joined to the core shaft 13. Specifically, the distal end of the coil body 15 is joined to the stranded wire 16 by a brazing material 12, and the proximal end of the coil body 15 is joined to the core shaft 13 by a brazing material 14.

Incidentally, the brazing material 12, the brazing material 14 and the brazing material 18 may use a material similar to that of the brazing material 2 and the brazing material 4 of the guidewire 1. Also, the coil body 15 is similar to the coil body 5 of the guidewire 1, and thus an outer diameter of the coil body 15 is larger than an outer diameter of the core shaft 13 at the position at which the coil body 15 is joined.

The bulge portion 19 is joined to the stranded wire 16 at a position spaced distally from the distal end of the core shaft 13 and the distal end of the coil body 15. An outer diameter of the bulge portion 19 is greater than an outer diameter of the core shaft 13 at a position at which the bulge portion 19 is joined to the core shaft 13. Incidentally, material of the bulge portion 19 is also similar to that of the bulge portion 9 of the guidewire 1.

An area of the coating agent 17 is illustrated by hatching in FIG. 4. As shown in FIG. 4, the coating agent 17 covers the core shaft 13, the coil body 15 and the bulge portion 19 from a distal end of the guidewire 10 (which is at a distal side of the distal end of the bulge portion 19) to the proximal side of the proximal end of the coil body 15 except for the interior of the coil body 15.

It is to ensure the flexibility of the distal end of the guidewire 10 not to allow the coating agent 17 to penetrate inside the coil body 15.

The coating agent 17 can cover the core shaft 13 all the way to the proximal end of the core shaft 13 on the proximal side of the proximal end of the coil body 15. Material of the coating agent 17 is similar to that of the coating agent 7 of the guidewire 1.

Because the guidewire 10 comprises a core shaft 13, a stranded wire 16 joined to a distal portion of the core shaft 13 so as to be located on a distal side of the core shaft 13 and including a plurality of wires, a coil body 15 having a distal end joined to the stranded wire 16 at a position spaced proximally from a distal end of the stranded wire 16 and a proximal end joined to the core shaft 13, a coating agent 17 covering an outer periphery of the stranded wire 16, the core shaft 13 and the coil body 15, and a bulge portion 19 joined to the stranded wire 16 at a position spaced distally from a distal end of the coil body 5, wherein the coating agent 17 covers the core shaft 13 and the coil body 15 from a distal side of the bulge portion 19 to a proximal side of the proximal end of the coil body 15 except for the interior of the coil body 15, it is possible to ensure flexibility of the coil body 15 and a distal portion of the guidewire 10 and prevent the coating agent 17 from peeling from the stranded wire 16, the core shaft 13 and the coil body 15 by the bulge portion 19 and the coil body 15.

The bulge portion 19 may be joined to the distal end of the stranded wire 16 so that the distal end of the stranded wire 16 does not protrude from the bulge portion 19. According to this configuration, it is possible to prevent the distal end of the stranded wire 16 from protruding from the coating agent 17 and damaging to the body tissue.

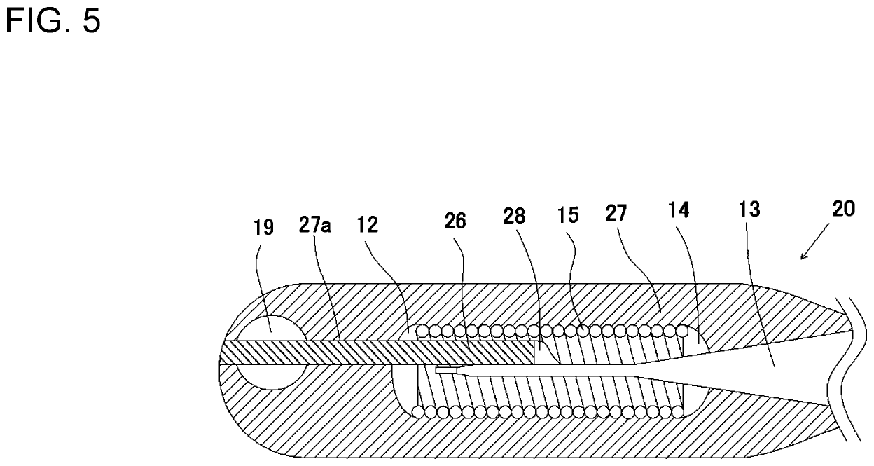

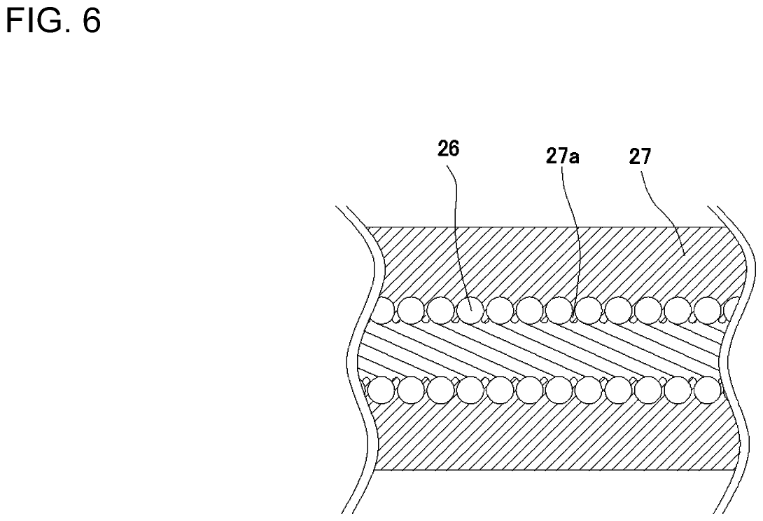

FIG. 5 is a longitudinal sectional view of a distal portion of a guidewire of the disclosed embodiments, and FIG. 6 is a longitudinal sectional view near a stranded wire of the guidewire of FIG. 5.

Incidentally, portions common to the guidewire 10 will be denoted by the same reference numerals, and descriptions of the portions will be omitted.

The guidewire 20 of the disclosed embodiments is different from the guidewire 10 with respect to the connecting state between the stranded wire and the coating agent. That is, a coating agent 27 penetrates between a plurality of wires included in a stranded wire 26 to form a protruding portion 27a that protrudes from a gap between adjacent ones of the plurality of wires included in the stranded wire 26.

The guidewire 20 increases bonding strength between the coating agent 27 and the stranded wire 26 by forming the protruding portion 27a. Further, because an innermost portion of the protruding portion 27a in the radial direction of the guidewire has a longitudinal thickness that is greater than a spacing between adjacent wires, it is possible to further prevent the coating agent 27 from peeling from the stranded wire 26.

As shown in FIG. 5, the stranded wire 26 includes a proximal end joined to a distal portion of the core shaft 13 by a brazing material 28 inside the coil body 15, and extends distal to a distal end of the core shaft 13 and a distal end of the coil body 15. Incidentally, the brazing material 28 may be a material similar to that of the brazing material 2 and the brazing material 4 of the guidewire 1.

Because the coating agent 27 penetrates between a plurality of wires included in the stranded wire 26 in at least a portion of the stranded wire 26 that is not inside the coil body 15, it is possible to further prevent the coating agent 27 from peeling from the stranded wire 26.

The bulge portion 19 may be joined to the distal end of the stranded wire 26 so that the distal end of the stranded wire 26 does not protrude from the bulge portion 19. According to this configuration, it is possible to prevent the distal end of the stranded wire 26 from protruding from the coating agent 27 and damaging to the body tissue.

1. A guidewire comprising:
a core shaft;
a coil body having a distal end that is joined to the core shaft at a position spaced proximally from a distal end of the core shaft;
a bulge portion joined to the core shaft at a position spaced distally from the distal end of the coil body; and
a coating agent covering an outer periphery of the core shaft and the coil body from a position distal to a distal end of the bulge portion to a position proximal to a proximal end of the core shaft except for an interior of the coil body.

2. A guidewire comprising:
a core shaft;
a stranded wire joined to a distal portion of the core shaft, including a plurality of wires, and extending distal to a distal end of the core shaft;
a coil body having a distal end that is joined to the stranded wire at a position spaced proximally from a distal end of the stranded wire, and that extends distal to the distal end of the core shaft;
a bulge portion joined to the stranded wire at a position spaced distally from the distal end of the coil body; and
a coating agent covering an outer periphery of the core shaft, the coil body, and the stranded wire from a position distal to a distal end of the bulge portion to a position proximal to a proximal end of the coil body except for an interior of the coil body.

3. The guidewire according to claim 2, wherein a proximal end of the stranded wire is joined to the distal portion of the core shaft inside the coil body.

4. The guidewire according to claim 3, wherein the proximal end of the stranded wire is joined to the distal portion of the core shaft at a position approximately halfway along a longitudinal length of the coil body.

5. The guidewire according to claim 2, wherein the coating agent comprises a protruding portion that penetrates from a gap between adjacent ones of the plurality of wires included in the stranded wire in at least a portion of the stranded wire that is not inside the coil body.

6. The guidewire according to claim 5, wherein an innermost portion of the protruding portion in a radial direction of the guidewire has a longitudinal thickness that is greater than a spacing between the adjacent wires.
